(19) United States
(12) Patent Application Publication
(10) Pub. No.: US 2022/0265576 A1
(43) Pub. Date: Aug. 25, 2022

(54) PHOSPHOLIPASE-A2 INHIBITORS FOR THE PREVENTION OF CANCER METASTASIS

(71) Applicant: COEGIN PHARMA AS, Trondheim (NO)

(72) Inventors: Berit Johansen, Trondheim (NO); Astrid Jullumstro Feuerherm, Trondheim (NO)

(21) Appl. No.: 17/629,585

(22) PCT Filed: Jul. 27, 2020
(86) PCT No.: PCT/EP2020/071168

§ 371 Date: Jan. 24, 2022

(30) Foreign Application Priority Data
Jul. 25, 2019 (GB) 1910644.2

(51) Int. Cl. A61K 31/121; A61P 35/00

(52) U.S. Cl. CPC A61K 31/121; A61P 35/00

(57) ABSTRACT

A compound of formula (I)

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a salt thereof;

for use in the prevention of metastasis in cancer, especially breast cancer.

This invention relates to the prevention of metastasis in cancer, especially breast cancer. In particular, the invention relates to the administration of certain polyunsaturated ketone compounds to a patient with pre-metastatic breast cancer and/or a cancer at high risk of metastasis with a view to preventing metastasis.

### BACKGROUND OF INVENTION

Breast cancer is the most common cancer affecting women, causing over half a billion deaths per year worldwide. Most of these patients die of metastasis, and not of the primary tumor. Thus, preventing cancers from developing metastatic lesions can save numerous patients' lives. Metastasis consists of multiple steps that cells from the primary tumor need to execute in order to complete the formation of a distant lesion. Stopping cells from undergoing one or more of these steps may represent a therapeutic opportunity to stop metastasis.

Inflammation and cancer are tightly connected Immunocompetent cells and tumorigenic cells may operate through the same signalling pathways and the inflammasome has emerged as a potential target to combat cancer. A fundamental attribute of the metastatic cancer cell is the ability to migrate through tissue. Inflammatory signalling is a central component of induction of the migratory phenotype in non-malign as well as malign conditions. A key regulatory step of inflammation is the formation of small auto- and paracrine bioactive lipids. Cytosolic phospholipase A2 α (cPLA2α or PLA2 GIVA, gene PLA2G4A) is a key enzyme in the formation of these and thus has a role in facilitating cancer cell migration. When cPLA2α hydrolyzes an intracellular membrane phospholipid that contains esterified arachidonic acid (AA), a free AA molecule and a lysophospholipid are generated. Further metabolization by downstream enzymes like cyclooxygenase 2 (COX-2) or lipoxygenases results in the generation of a spectrum of lipid-derived signalling mediators, such as prostaglandins and leukotrienes. Activity of pathways such as Raf/Ras/MEK/Erk may lead to activation of cPLA2α by phosphorylation and translocation to membranes is stimulated by increased intracellular Ca²⁺.

Cytosolic PLA2α signalling can interact with oncogenic pathways, such as PI3K/Akt pathway and Nuclear Factor Kappa B (NF-KB). Accordingly, cPLA2α has been implicated in tumorigenesis, cancer progression, and metastasis in several cancer forms, including breast cancer. In breast cancer, high cPLA2α expression levels are associated with the more aggressive, triple negative phenotypes and lower survival rates, suggesting a role in breast cancer metastasis. This effect may be mediated by prostaglandin E2 (PGE2), which is a known tumorigenic and pro-migratory eicosanoid produced from AA.

Inhibition of cPLA2α has for long been proposed as a promising anti-inflammatory target. Furthermore, cPLA2α also has shown anti-tumorigenic and anti-angiogenic effects in cancer. We have previously shown that various cPLA2α inhibitors efficiently target inflammation, tumor progression and angiogenesis, in vitro and in vivo (Anthonsen M W, Solhaug A, Johansen B. Functional coupling between secretory and cytosolic phospholipase A2 modulates tumor necrosis factor- alpha- and interleukin-1beta-induced NF-kappa B activation. J Biol Chem 2001;276:30527-36).

The inventors have now found that certain selective inhibitors of cPLA2α can reduce the migration of a metastatic cancer cell.

The compounds of the invention are not new and have previously been disclosed for the treatment of various conditions (see for example WO2012/028688, and WO2010/139482). WO2015/181135 describes the use of certain polyunsaturated ketones for treating skin cancer. In WO2017/157955, there is a discussion of the combination of a compound of the invention with a co-agent and the suggestion that such a combination is interesting in the treatment of cancers including breast cancer. Nowhere before however has it been demonstrated that the compounds of the invention can prevent or at least inhibit metastasis in cancer. In particular, prevention of metastasis can be effected using the compounds of the invention as the sole active anti-metastatic component in a pharmaceutical. Prevention would not require a combination therapy although a second pharmaceutical might be used to treat the underlying cancer. The present invention is therefore ideally targeted towards patients with breast cancer but without metastasis and/or patients with cancers at high risk of metastasis.

### SUMMARY OF INVENTION

Viewed from one aspect the invention provides a compound of formula (I)

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a salt thereof;

for use in the prevention of metastasis in cancer, especially breast cancer.

Viewed from another aspect the invention provides a method of preventing metastasis in cancer, especially breast cancer, comprising administering to a patient in need thereof, e.g. human, an effective amount of a compound of formula (I):

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a salt thereof.

Viewed from another aspect the invention provides use of a compound of formula (I) or a salt thereof as hereinbefore described for use in the manufacture of a medicament for preventing metastasis in cancer, especially breast cancer.

Viewed from another aspect the invention provides a compound of formula (I)

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a salt thereof;

for use in a method of prevention of metastasis in breast cancer comprising administering said compound to a patient with pre-metastatic breast cancer.

### DETAILED DESCRIPTION OF THE INVENTION

The present invention aims to prevent metastasis in cancer. The invention relies on the administration to a patient, such as a patient with pre-metastatic breast cancer or a cancer at high risk of metastasis, a compound of formula (I).

### Compound (I)

The invention relies on the therapeutic combination of a compound of formula (I). The compound of formula (I) is

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a salt thereof.

The group R preferably comprises 5 to 9 double bonds, preferably 5 or 8 double bonds, e.g. 5 to 7 double bonds such as 5 or 6 double bonds. These bonds should be non-conjugated. It is also preferred if the double bonds do not conjugate with the carbonyl functionality.

The double bonds present in the group R may be in the cis or trans configuration however, it is preferred if the majority of the double bonds present (i.e. at least 50%) are in the cis configuration. In further advantageous embodiments all the double bonds in the group R are in the cis configuration or all double bonds are in the cis configuration except the double bond nearest the carbonyl group which may be in the trans configuration.

The group R may have between 10 and 24 carbon atoms, preferably 12 to 20 carbon atoms, especially 17 to 19 carbon atoms.

Whilst the R group can be interrupted by at least one heteroatom or group of heteroatoms, this is not preferred and the R group backbone preferably contains only carbon atoms.

The R group may carry up to three substituents, e.g. selected from halo, C1-6 alkyl e.g. methyl, or C₁₋₆ alkoxy. If present, the substituents are preferably non-polar, and small, e.g. a methyl group. It is preferred however, if the R group remains unsubstituted.

The R group is preferably an alkylene group.

The R group is preferably linear. It preferably derives from a natural source such as a long chain fatty acid or ester. In particular, the R group may derive from arachidonic acid, eicosapentaenoic acid or docosahexaenoic acid.

Thus, viewed from another aspect the invention employs a compound of formula (I′)

R-L-CO—X (I′)

wherein R is a C₁₀₋₂₄ unsubstituted unsaturated alkylene group said group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group or a salt thereof.

Ideally R is linear. R is therefore preferably an unsaturated C₁₀₋₂₄ polyalkylene chain.

The linking group L provides a bridging group of 1 to 5 backbone atoms, preferably 2 to 4 backbone atoms between the R group and the carbonyl, such as 2 atoms. The atoms in the backbone of the linker may be carbon and/or be heteroatoms such as N, O, S, SO, or SO₂. The atoms should not form part of a ring and the backbone atoms of the linking group can be substituted with side chains, e.g. with groups such as C₁₋₆ alkyl, oxo, alkoxy, or halo.

Preferred components of the linking group are —CH₂—, —CH(C₁₋₆alkyl)—, —N(C₁₋₆alkyl)—, —NH—, —S—, —O—, —CH═CH—, —CO—, —SO—, —SO₂—which can be combined with each other in any (chemically meaningful) order to form the linking group. Thus, by using two methylene groups and an —S—group the linker —SCH₂CH₂—is formed. It will be appreciated that at least one component of the linker provides a heteroatom in the backbone.

The linking group L contains at least one heteroatom in the backbone. It is also preferred if the first backbone atom of the linking group attached to the R group is a heteroatom or group of heteroatoms.

It is highly preferred if the linking group L contains at least one —CH₂—link in the backbone. Ideally the atoms of the linking group adjacent the carbonyl are —CH₂—.

It is preferred that the group R or the group L (depending on the size of the L group) provides a heteroatom or group of heteroatoms positioned α, β, γ, or δ to the carbonyl, preferably β or γ to the carbonyl. Preferably the heteroatom is O, N or S or a sulphur derivative such as SO.

Highly preferred linking groups L therefore are —NH₂CH₂, —NH(Me)CH₂—, —SCH_{2—}, or —SOCH₂—.

The linking group should not comprise a ring.

Highly preferred linking groups L are SCH₂, NHCH₂, and N(Me)CH₂.

Viewed from another aspect the invention employs a compound of formula (II)

R-L-CO—X (II)

wherein R is a linear C₁₀₋₂₄ unsubstituted unsaturated alkylene group said group comprising at least 4 non-conjugated double bonds;

L is —SCH₂—, —OCH₂—, —SOCH₂, or —SO₂CH₂—; and

X is an electron withdrawing group or a salt thereof.

The group X is an electron withdrawing group. Suitable groups in this regard include O—C₁₋₆ alkyl, CN, OCO₂—C₁₋₆ alkyl, phenyl, CHal₃, CHal₂H, CHalH₂ wherein Hal represents a halogen, e. g. fluorine, chlorine, bromine or iodine, preferably fluorine.

In a preferred embodiment the electron withdrawing group is CHal3, especially CF₃.

Thus, preferred compounds of formula (I) are those of formula (III)

R-Y1-Y2-CO—X (III)

wherein R and X are as hereinbefore defined;

Y1 is selected from O, S, NH, N(C₁₋₆-alkyl), SO or SO₂ and

Y2 is (CH₂)n or CH(C₁₋₆ alkyl); or

where n is 1 to 3, preferably 1.

More, preferred compounds of formula (I) are those of formula (IV)

R-Y1-CH₂—CO—X (IV)

wherein R is a linear C₁₀₋₂₄ unsubstituted unsaturated alkylene group said group comprising at least 4 non-conjugated double bonds;

X is as hereinbefore defined (e.g. CF₃); and

Y1 is selected from O, S, SO or SO₂.

More, preferred compounds of formula (I) are those of formula (V)

R—S—CH₂—CO—CF₃ (V)

wherein R is a linear C₁₀₋₂₄ unsubstituted unsaturated alkylene group said group comprising at least 4 non-conjugated double bonds.

Highly preferred compounds for use in the invention are depicted below:

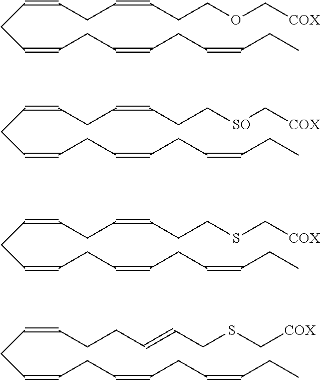

where X is as hereinbefore defined such as CF₃.

The following compounds are highly preferred for use in the invention:

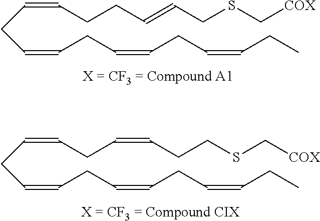

It will be appreciated that the compound of formula (I) may be administered in the form of a pharmaceutical composition. It will be appreciated that the compound of formula (I) may be administered in the form of a pharmaceutical acceptable salt is required.

### Cancer

The invention preferably targets breast cancer although other cancers at risk of metastasis could also be targeted. In order to prevent metastasis, the patient to which the compound of the invention is administered should be one whose cancer has not metastasised. The compound of the invention may be the sole pharmaceutical administered to the patient in order to prevent metastasis. However, the patient may also be subject to conventional drug regimens to treat the underlying cancer.

Viewed from another aspect therefore the invention may provide a pharmaceutical composition for simultaneous, sequential or separate use comprising a kit comprising a first composition comprising a compound (I) as defined in claim 1 and a pharmaceutically-acceptable diluent or carrier, and a second composition comprising a compound to treat the underlying cancer, e.g. breast cancer, and a pharmaceutically-acceptable diluent or carrier.

This invention targets breast cancer, more specifically non metastatic breast cancer.

By prevention is meant (i) preventing or delaying the appearance of clinical symptoms of the disease developing in a mammal.

The benefit to a subject to be treated is either statistically significant or at least perceptible to the patient or to the physician. In general a skilled person can appreciate when “prevention” occurs.

The composition of the invention can be used on any animal subject, in particular a mammal and more particularly to a human or an animal serving as a model for a disease (e.g., mouse, monkey, etc.).

In order to prevent a disease an effective amount of the active compound needs to be administered to a patient. A “therapeutically effective amount” means the amount of a compound that, when administered to an animal for treating a state, disorder or condition, is sufficient to effect such treatment. The “therapeutically effective amount” will vary depending on the compound, the disease and its severity and the age, weight, physical condition and responsiveness of the subject to be treated and will be ultimately at the discretion of the attendant doctor.

It may be that to prevent metastasis according to the invention that the compound has to be readministered at certain intervals. Suitable dosage regimes can be prescribed by a physician.

The compound of the invention is typically administered in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on.

It will be appreciated that pharmaceutical compositions for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients. The compositions of the invention could also be formulated as nanoparticle formulations.

However, for the treatment of cancer, the composition of the invention will preferably be administered orally or by parenteral or intravenous administration, such as injection. The composition may therefore be provided in the form of an tablet or solution for injection.

The pharmaceutical composition comprising the compound of formula (I) may contain from 0.01 to 99% weight-per volume of the active material. The therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

It is within the scope of the present invention to administer the compound described herein to a subject along with one or more anti-proliferative compounds and particularly those known to be used in anti-cancer therapies. Non-limiting examples include aromatase inhibitors, anti-estrogens, topoisomerase I or II inhibitors microtubule active compounds, alkylating compounds, histone deacetylase inhibitors, and cyclooxygenase inhibitors such as those disclosed in WO2006/122806 and references cited therein Choice of whether to combine a compound of the invention with one or more of the aforementioned anti-cancer therapies will be guided by recognized parameters known to those of skill in the field, including the particular type of cancer being treated, the age and health of the subject, etc.

The invention is described further below with reference to the following non-limiting examples and figures.

### BRIEF DESCRIPTION OF THE FIGURES

FIG. 1. Response to inhibitor CIX and expression of cPLA2α in 67NR and 4T1. a) Image showing bonds for p-cPLA2α, cPLA2α, and beta-actin from baseline cell line samples, on the same membrane. b) Box-and-whiskers plot (min-max) of total cPLAα protein normalized to beta-actin. c) Box-and-whiskers plot (min-max) of phosphorylated cPLAα (p-S505) protein normalized to beta-actin. d) Viability curve based on XTT metabolization. Points represent grand mean of n=3 experiments with n=6 technical replicates per condition. IC50 values (48 h) are given in μM. *p<0.05 4T1 vs 67NR at same concentration. e) Proliferation curve based on EdU incorporation. Points represent grand mean of n=3 experiments with n=6 technical replicates per condition. IC50 values (24 h) are given in μM. *p <0.05 4T1 vs 67NR at same concentration. f) PGE2 levels in 67NR and 4T1 at 6 h and 24 h of treatment. The box-and-whiskers plot (min-max) is based on n=3 experiments with n=3 technical replicates per condition. *p<0.05 4T1 vs 67NR.

FIG. 2. CIX inhibits migration in 4T1, but not 67NR. a) Cell index related to vehicle control at end of 24 h treatment. Bars show percentage mean ±SD of 3 experiments, normalized to vehicle control within experiment. n=4 technical replicates per experiment b) Cell index related to vehicle control at end of 48 h treatment. Bars show percentage mean ±SD of 3 experiments, normalized to vehicle control within experiment. n=2-4 technical replicates per experiment. c) Cell index (CI) curves for 67NR and 4T1 over 24 h. Each curve shows mean CI±SD of n=4 technical replicates. Representative experiment of n=3 distinct experiments. d) Cell index curves for 4T1 over 48 h. Each curve shows mean CI±SD of n=4 technical replicates for CIX treated groups and Norm Ctrl, n=2 technical replicates for Pos Ctrl and Neg Ctrl. Representative experiment of n=3 distinct experiments. a-d) Norm Ctrl: Normal (vehicle) control. Pos Ctrl: Positive control (high chemoattractant). Neg Ctrl: Negative control (low chemoattractant). *p<0.05 vs. 4T1 Norm Ctrl, #p<0.05 vs. 67NR Norm Ctrl.

FIG. 3. Gene clusters of top-ranked GO-terms. Network of associated CIX-affected gene products were generated using STRING using only curated databases as active interaction sources. Light green nodes are upregulated, whereas dark green nodes are downregulated in 4T1 cells in response to CIX (15 μM, 24 hrs). TLR signalling stand out as a key cluster in this network.

FIG. 4. Hypothesized effects of CIX in TLR signalling in 4T1 cells. By reduced signalling through TLR3, TLR4, TLR9 and NF-kB (rel), it is likely that cPLA2α inhibition affect cancer microenvironment and reduce both cancer cell viability, migration and the level of inflammation. Such altered cancer cell microenvironment may suggest that cPLA2α regulates many aspects of cancer cell biology and thus serves as an attractive target for metastatic cancer. Dark green nodes represent genes found to be downregulated, light green nodes represent upregulated genes. Grey and white nodes represent genes assumed to be affected or without known effects, respectively.

### EXAMPLES

The following compound is used in the examples which follow.

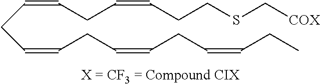

### Cell Culture

Two isogenic cell lines, stemming from a single spontaneous mammary triple-negative tumor, were used for all experiments. While both cell lines effectively establish primary tumors, 67NR cells will not metastasize, and 4T1 cells may form metastatic lesions in lung, brain, lymph nodes, bone, and liver. Cells were kept in vented flasks in a humidified atmosphere at 5% CO2, 37° C., and stock flasks routinely split 1:8-1:10 (67NR) and 1:15-1:20 (4T1) twice a week using 0.25% trypsin/EDTA. Passage numbers were between 15 and 55. Culture medium was Dulbecco's Modified Eagle Medium (DMEM)/4.5 mg/ml glucose (Gibco, Thermo Fisher Scientific, Waltham, USA) with 10% Fetal Bovine Serum (PBS; Gibco), 0.1 mg/ml penicillin-streptomycin, and 1 μg/ml amphotericin. Prior to experiments, stock cells were synchronized once or twice by seeding into a new flask with a split ratio of 1:2-1:5, incubated overnight, trypsinized and seeded into wells in growth culture medium. Seeded cells were allowed to attach and grow overnight before treatment. Dimethyl sulfoxide (DMSO) was used as vehicle for the inhibitor in all experiments.

### XTT Assay

The XTT (2,3-Bis-[2-methoxy-4-nitro-5-sulfophenyl[2H-tetrazolium-5-carboxanilide) assay is a colorimetric assay in which the tetrazolium salt is converted in mitochondria of metabolically active cells, hence the signal is proportional to viable cells. Cells were seeded in 96-well flat-bottom plates, with cell numbers optimized to get 60-80% confluency at start of experiment. After overnight incubation, growth medium was removed and the experiment was performed in serum-free medium. An incubation time of 48 h following addition of inhibitor was chosen for readout in order to detect differential effects in subsequent assays. The TACS XTT Cell Proliferation/Viability Assay (Trevigen, Gaithersburg, Md., USA) was performed according to manufacturer's manual, with 1.5-2 h incubation. Readout at 490 nm with subtraction of background reading at 655 nm was carried out on an iMark Microplate Reader (BIO RAD, Hercules, Calif., USA). Results are given as % viability based on average of sextuplets compared to intra-assay vehicle controls; two-tailed Student's t-test was used to evaluate the statistical significance. IC50, the concentration which reduced signal to 50% of controls, was calculated using non-linear fit of [Inhibitor] vs. response—Variable slope (four parameters), in GraphPad 7 for Windows.

### Proliferation Assay

A Click-IT EdU microplate assay (Invitrogen, Thermo Fisher Scientific) was used to determine the effect of CIX concentration on proliferation. Cells were seeded as described for the XTT assay. EdU (final concentration 3-5 μM) was added to wells directly after applying treatments, and cells were incubated for 24 h before performing the assay according to the manufacturer's manual. Readout was carried out after 24 h EdU exposure on a POLARstar Omega plate reader with excitation/emission at 540/580 with orbital averaging. IC50 was calculated in the same way as for XTT data.

### Protein Extraction

To generate protein lysate for subsequent assays, cells were seeded in 6-well plates to obtain 60-80% confluency 24 h post-seeding. After treatment, supernatant was removed for subsequent ELISA, and proteins were extracted in ice-cold RIPA buffer containing 2% cOmplete™ Protease Inhibitor Cocktail, 1% Phosphatase Inhibitor Cocktail 2, and 1% Phosphatase Inhibitor Cocktail 3 (all Merck KGaA, Darmstadt, Germany). The lysate was agitated for 30 min at 4° C., sonicated for 1.5 min, and centrifuged for 20 min at 4° C., 12 000 rpm. The protein supernatant was stored at −80° C.

### Western Blotting

Protein concentration was quantified using the Pierce BCA assay (Thermo Fisher Scientific). To compare the levels of cPLA2α in 4T1 and 67NR, protein lysates were separated by sodium dodecyl sulfate polyacrylamide gel electrophoresis and identified and semi-quantitated using the Western blot technique with IR-labeled secondary antibodies. All Bolt reagents were from Invitrogen, Thermo Fisher Scientific. The protein lysate was heated at 70° C. for 10 min in 4x Protein Loading Buffer (LI-COR Biosciences, Lincoln, Nebr., USA) and Bolt Sample Reducing Agent, before 5 μg protein was loaded onto a Bolt Bis-Tris 4-12% gel and separated at 200 V for 70 min, using Bolt MOPS SDS running buffer and Bolt Antioxidant in front chamber and Chameleon Duo Pre-stained Protein Ladder (LI-COR Biosciences) for size marking. Wet transfer to PVDF membrane was done at 20 V for 2 h, using Bolt Transfer buffer. Following blocking with Odyssey Blocking buffer TBS (LI-COR Biosciences) overnight at 4° C. and cutting at 50 kDa mark for separate incubation with anti-beta actin antibodies, the membranes were incubated with goat anti-phospholipase A2 antibody (ab104252, Abcam; 1:3000) and rabbit anti-phospholipase A2 (phospho S505) antibody (ab53105 from Abcam, Cambridge, UK; 1:3000), or mouse anti-beta actin (ab8226 from Abcam; 1:25 000), overnight at 4° C. Secondary fluorescent antibody conjugates (IRDye® 800CW donkey anti-Goat IgG and 680CW Goat anti-rabbit IgG, or IRDye® 800CW Goat Anti-mouse IgG; LI-COR Biosciences) were applied at 1:30 000 dilution in blocking buffer/0.2% Tween-20/0.01% SDS. Imaging was performed on an Odyssey Clx (LI-COR Biosciences) in the 700 and 800 channels at the Auto setting. Quantification of bands were performed by subtracting local background and the signal was normalized to beta actin in Image Studio Lite (LI-COR Biosciences). Two-tailed Student's t-test was used to evaluate the statistical significance between groups.

### Enzyme Immunoassay

A sensitive, quantitative enzyme immunoassay specific for the metabolite PGE2 was used as a proxy to assess cPLA2α activity. Supernatant from cells used to make protein lysates for RPPA were spun at 1500 rpm for 10 min at 4° C. and stored at −80° C. After thawing, supernatants were spun at 2000 rpm 5 min 4° C., diluted 1:50 in DMEM, and analyzed in duplicate using a 96-well PGE2 EIA Kit Monoclonal (Cayman Chemical Company, Ann Arbor, Mich., USA). Analysis was performed using the 4 Parameter Logistic Curve function in the MyAssays Prostaglandin E2-Monoclonal online analysis tool https://www.myassays.com/prostaglandin-e2-monoclonal.assay. Two-tailed Student's t-test was used to evaluate the statistical significance (p<0.05).

### Migration Assay

Cell migration was assessed using the xCELLigence® DP system (Roche Diagnostics GmbH, Germany). In this assay, cells are added to a membrane surface in the upper part of a CIM 16 plate, and cells can migrate through the membrane to the lower wells as a response to a chemoattractant signal. Gold electrodes are attached underneath the membrane, and cells in contact with electrodes reduce the conductivity. The change is interpreted inversely as the Cell Index (CI). Here, 5.0×10⁴ cells/well were seeded in 0.5% FBS/DMEM in upper wells containing inhibitor or vehicle (DMSO). As a chemoattractant, 5 or 10% FBS/DMEM was added to lower wells for normal or positive controls, respectively, with 0.5% FBS used to control for increased background migration (negative controls) in 48 h experiments. The plate was scanned every 15 min for 24 h or 48 h. Plotting of CI curves was carried out using RTCA Software 1.2 supplied with the instrument. For relative quantification, all technical replicates of a treatment were normalized to the intra-experiment normal control. Two-tailed Student's t-tests were performed to find significant differences between groups (p<0.05).

### RNA Sequencing

RNA for sequencing of the global mRNA transcriptome was isolated from 4T1 or 67NR cells treated with 15 μM CIX or vehicle (n=4 biological replicates) for 24 h using the RNeasy Mini kit (Qiagen, Limburg, The Netherlands) according to manufacturer's manual. RNA concentration and purity were quantified using a NanoDrop 1000 (Thermo Scientific, Waltham, USA). An Agilent Bioanalyzer (Agilent, Santa Clara, USA) was used to measure RNA Integrity Numbers (RIN), which were between 9.7-10. A library was prepared using an Illumina TruSeq Stranded mRNA kit (Illumina, San Diego, USA), sequencing was done on an Illumina NextSeq using a NS500HO flow cell with 75 bp reads, and quality control of raw sequences was performed with the FastQC application https://www.bioinformatics.babraham.ac.uk/projects/fastqc/.

### Gene Expression Analysis

Transcript expression values were generated by quasi-alignment using salmon http://salmon.readthedocs.io/en/latest/salmon.html and the Ensembl (GRCm38) mouse genome. Aggregation of transcript to gene expression was performed using tximport. Gene expression values with TPM (transcript per million) below one in more than three samples were filtered out before differential expression was assessed by limma-voom linear model. Significance was defined by a Benjamini-Hochberg multiple comparison-adjusted p-value<0.001. Enrichr, a tool for gene enrichment analysis, was employed to find gene enrichment signatures for these genes within the Gene Ontology Biological Process 2018 database. Significance was denoted as p<0.05, corrected for multiple testing by the Benjamini-Hochberg procedure. Clustering of the proteins encoded by the genes included in the top-ranked GO terms, were performed using STRING version 11.0 and modified using Adobe Illustrator.

### LIST OF ABBREVIATIONS

COX-2: Cyclooxygenase 2, DMEM: Dulbecco's Modified Eagle Medium, DMSO: Dimethyl sulfoxide, FBS: Fetal Bovine Serum, IFN: Interferon, PGE2: Prostaglandin E2, TLR: Toll-like receptor

### RESULTS AND DISCUSSION

### 4T1 Cells Express Higher Levels of cPLA2α than 67NR Cells

The 4T1 model consists of several isogenic cell lines that arise from the same murine breast tumor, but have widely different metastatic abilities. We used two cell lines from the 4T1 model, 67NR and 4T1, that are non-metastatic and highly metastatic, respectively, to investigate whether cPLA2α expression and activity were involved in the metastatic phenotype.

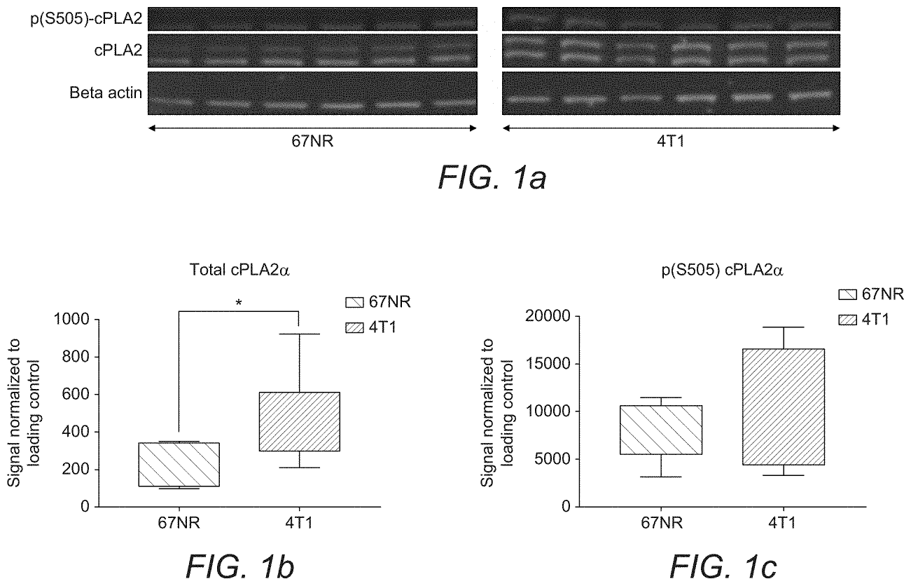

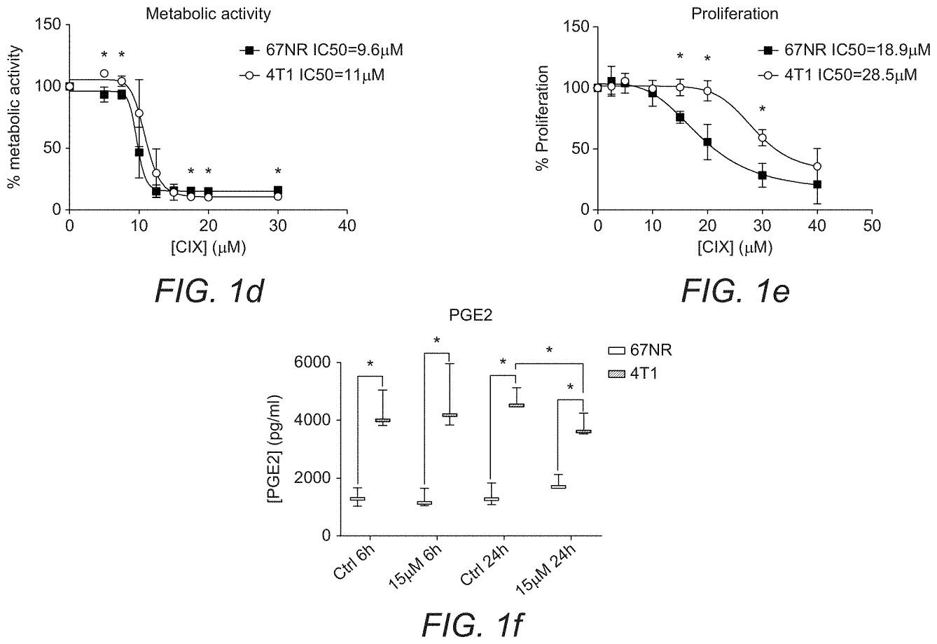

Initially, the baseline expression of cPLA2α protein in both cell lines was determined by western blotting (FIG. 1a-c). Two bands were observed for total protein detection, as is often observed for cPLA2α blots with the slightly larger band being more intense in 4T1 (FIG. 1a). This band showed up consistently across all samples (including a human cell line control sample) and replicates and was not removed with calf intestinal phosphatase or lambda phosphatase, in contrast to S505 bands (data not shown). 4T1 cells expressed 1.95-fold more cPLA2α protein than 67NR cells (FIG. 1b). Phosphorylation of cPLA2α on S5505 typically corresponds to activation of cPLA2α, and a non-significant tendency of higher phosphorylation status was seen in 4T1 (FIG. 1c). In addition to more total protein, this may imply that 4T1 has higher basal activity in pathways involving cPLA2α.

### 67NR and 4T1 Cells Show Different Sensitivity to CIX Treatment

Next, we employed the cPLA2α inhibitor CIX to assess the effect of cPLA2α inhibition in our model. In order to establish a dose-response relationship of CIX and viability, we tested a range of doses on both cell lines in metabolic activity (XTT) and proliferation (EdU incorporation) assays. Both cell lines displayed a dose-dependent relationship between CIX concentration and assay output (FIG. 1d-e). In the XTT assay, 67NR and 4T1 displayed IC50 values of 9.6 μM and 11 μM, respectively, after 48 h. The difference was not statistically significant (p<0.05) at 10 μM, likely due to higher interassay variation, there was however a significant difference at lower and higher doses. For proliferation, the IC50 values were 18.9 μM and 28.5 μM (67NR and 4T1, respectively) after 24 h.

The ability of the cPLA2α inhibitor CIX to impede proliferation differed greatly in the two cell lines. Hence, lower mitochondrial metabolism did not directly correspond to lowered proliferation.

### cPLA2α Inhibition Reduces PGE2 Production in 4T1 Cells

PGE2, a tumorigenic and pro-migratory metabolite downstream of cPLA2α and COX-2, was measured in both cell lines after treatment with 7.5 and 15 μM CIX for 6 and 24 h (FIG. 1f). PGE2 levels were significantly higher in 4T1 compared to 67NR at both time points (3.25-fold and 3.38-fold difference at 6 h and 24 h, respectively; FIG. 1f), reflecting higher expression and activity of cPLA2α, which may also be related to increased migratory potential of metastatic cells. Treatment with 15 α M CIX significantly lowered PGE2 in 4T1 after 24 h (0.81-fold), whereas no reduction was observed in 67NR. This may imply that CIX normalizes PGE2 production but does not block it. Other PLA2s may also release AA and thereby contribute to PGE2 levels.

### cPLA2α Inhibition Impedes Migration in 4T1 Cells

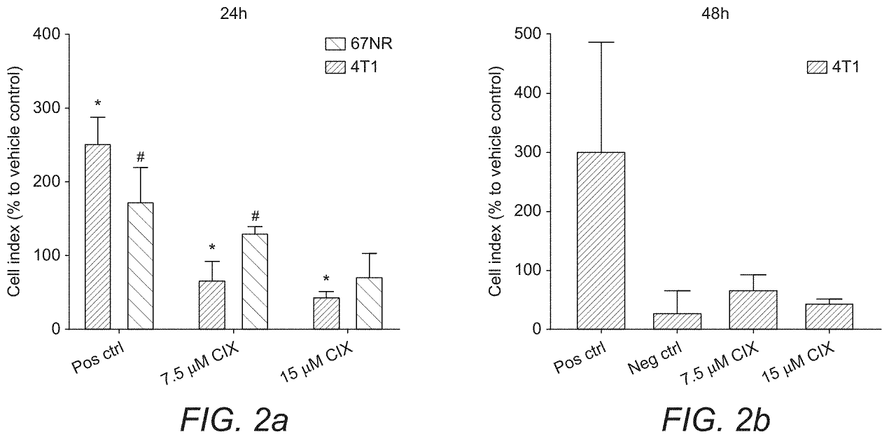

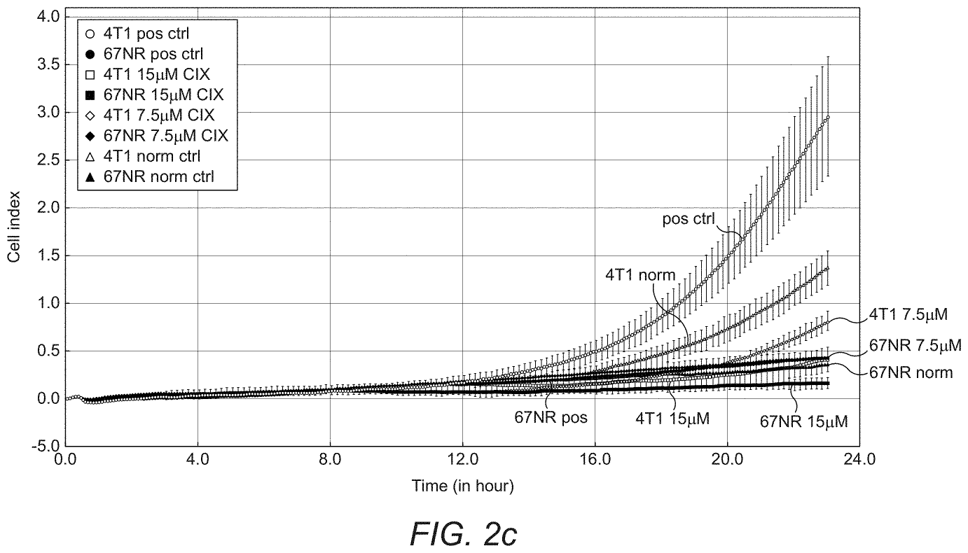

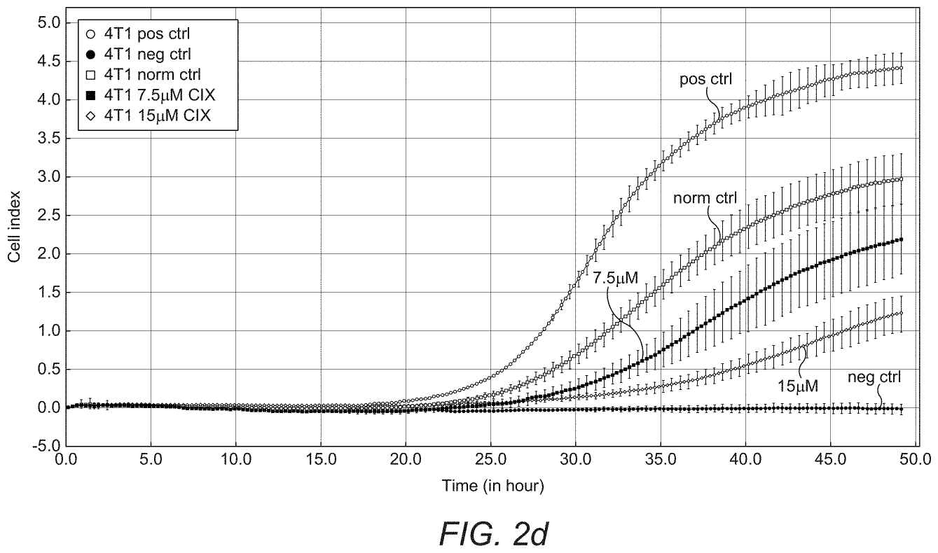

We next evaluated the effect of CIX on migration at dose levels that did not affect proliferation. The migratory capacity of the two cell lines were different, with 4T1 showing a 5-fold higher basal migration compared to 67NR after 24 h in normal controls (FIG. 2a, c).
Significant induction of migration in the positive controls (with increased levels of chemoattractant PBS) was demonstrated in both cell lines (FIG. 2b, d). After 24 h, 7.5 or 15 μM CIX did not impair migration in 67NR. In contrast, CIX significantly inhibited migration in 4T1 in a dose dependent manner in both 24 h and 48 h experiments. For the 4T1 cells, the reduction of PGE2 and migration at the same CIX dose and time point may indicate that cPLA2α and PGE2 plays a role in the increased migratory capacity of 4T1 relative to 67NR.

### Transcriptomal Effects of cPLA2α Inhibition include Toll-Like Receptors and Type I Interferon Pathways

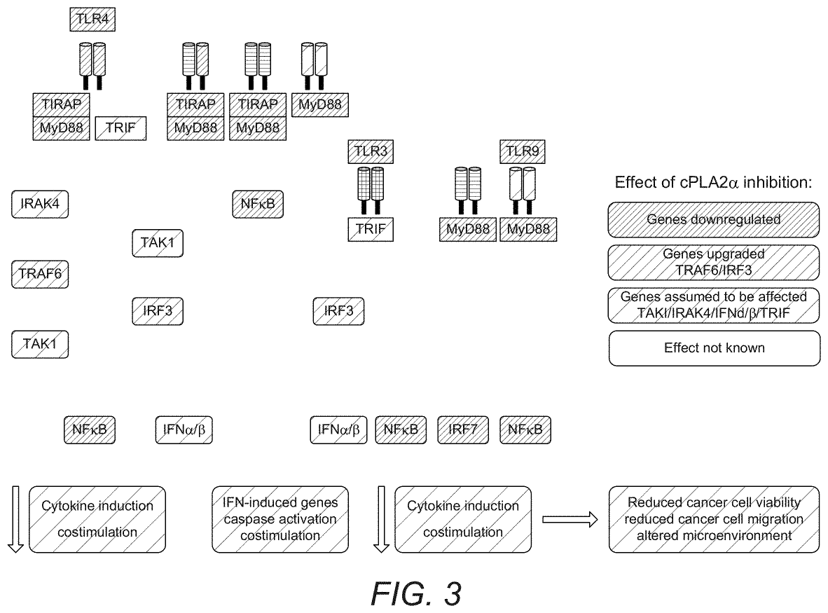

In order to investigate if the anti-migratory effect of CIX on 4T1 cells could be reflected in the transcriptome, we next performed a global gene expression analysis using RNA sequencing. In response to 15 μM CIX, 2887 genes were differentially expressed compared to untreated controls. To characterize the molecular responses affected by CIX treatment, the data set with differentially expressed genes were analyzed in Enrichr. The key GO Biological Processes identified were related to type I interferon (IFN-I) and TLR signalling, RNA splicing, and cell cycle regulation (Table 1). Many of the genes associated with these processes were downregulated in CIX treated cells compared to control cells. Using STRING, we next made an interaction network with the genes in the top-ranked GO terms that were regulated by CIX treatment that was linked to TLR and IFN-I signalling. This gave a network with several clusters, based on evidence of interaction between the proteins that are products of these genes (FIG. 3).

One cluster appearing in our interaction network contained the TLRs, Tlr3, Tlr4 and Tlr9 and the adaptor proteins Tirap and Myd88, all significantly less expressed at the gene level in CIX treated 4T1 cells compared to control cells. Furthermore, the TLR9-regulated transcription factor Irf7, inducing several IFN-α genes and cytokines, appeared in this cluster, and was downregulated in response to CIX. In contrast, the TLR3-regulated transcription factor IRF3 was upregulated. TLR stimulation activates NF-κB, MAPKs, Jun N-terminal kinases (JNKs), p38, and ERKs, as well as interferon regulatory factors such as IRF3/7, in turn regulating the production of inflammatory cytokines. Another prominent cluster contained the INF-I signalling pathway components STAT2, STAT6, IRF9, TYK2 and IFNAR2. Our data suggests that Myd88-dependent TLR signalling is reduced in response to CIX.

TLRs are central in recognition of invading microorganisms or internal damaged tissues, leading to an inflammatory response. TLRs are also central in cancer, including breast cancer, and exert contrasting effects on immune cells versus cancer cells. Using cPLA2α inhibitors, we have previously shown that cPLA2α regulates TLR2-induced PGE2 and pro-inflammatory gene expression in synoviocytes. Both TLR4 and TLR9 signalling pathways are associated with increased migration in breast cancer. A study from Wu et al. showed that the expression levels of TLR4 and MyD88 were significantly increased in breast tumors compared with normal breast tissue. The expression levels of TLR4 and MyD88 were also positively correlated with the metastatic potential of breast cancer cells and tumors.

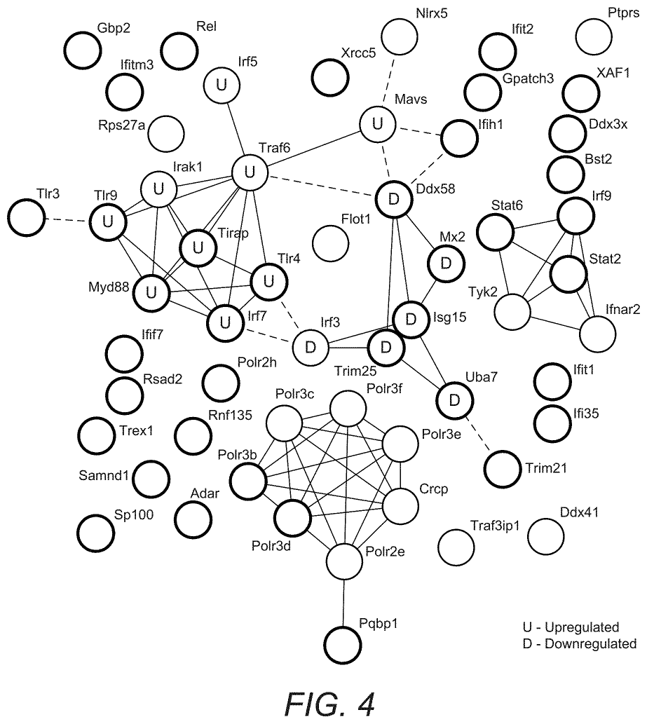

IFN-Is are induced by TLRs, which are pattern recognition receptors playing a pivotal role in innate immunity and cancer. IFN-Is, like TLRs, are emerging as double-edged swords in cancer. The role of IFN-Is in cancer has generally been considered beneficial by promoting T cell responses and preventing metastasis. On the other hand, IFN-Is may also has a negative role by promoting negative feedback and immunosuppression. IFN-I signalling may be a key driver of immune dysfunction in some cancers. Studies have shown an upregulation of the IFNα pathway in inflammatory breast cancer, and breast cancer tumors with high expression of interferon-response genes are shown to be associated with a significantly shorter overall survival and metastasis free survival. In our study, Stat2, Irf9, and Stat6 are significantly less expressed in CIX treated cells compared to control cells, suggesting that cPLA2α inhibition interferes with IFN-I signalling in 4T1 cells. However, no IFN's were themselves found to be regulated by CIX. Loss of STAT2 is earlier associated with decreased proliferation and migration in triple negative breast cancer.
Together, this implies that cPLA2α inhibition, via reduction of PGE2 and TLRs and by interfering with IFN-I signalling, reduces migration in highly metastatic 4T1 cells (FIG. 4).

Overall, our findings confirm that treatment with a low-molecular cPLA2α inhibitor reduces the viability of murine breast cancer cell lines. The metastatic 4T1 cell line had higher baseline cPLA2 activity and was less sensitive to CIX than the non-metastatic 67NR cell line metabolically, but more sensitive for the anti-migratory effect. cPLA2α inhibition reduced PGE2 production and blocked migration in the 4T1 cells. Gene expression analysis could not fully explain the anti-migratory effects but implied the involvement of TLR signalling and IFN-I signalling.

The selective cPLA2α inhibitor, CIX, specifically impedes migration of metastatic 4T1 cells. A comprehensive high throughput analysis at the transcriptome level in 4T1 cells indicates that cPLA2α inhibition affects TLR signalling and type I interferons.

1. A compound of formula (I)
R-L-CO—X (I)
wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;
L is a linking group forming a bridge of 1 to 5 atoms between the R group and the carbonyl CO wherein L comprises at least one heteroatom in the backbone of the linking group; and
X is an electron withdrawing group; or a salt thereof;
for use in the prevention of metastasis in cancer, especially breast cancer.

2. A compound for use as claimed in any preceding claim wherein X is CHal₃, preferably CF₃.

3. A compound for use as claimed in any preceding claim wherein in formula (I) R is a linear unsubstituted C₁₀₋₂₄ unsaturated alkylene group comprising at least 4 non-conjugated double bonds.

4. A compound for use as claimed in any preceding claim wherein L is —SCH₂—.

5. A compound for use as claimed in any preceding claim wherein said compound of formula (I) has the formula: 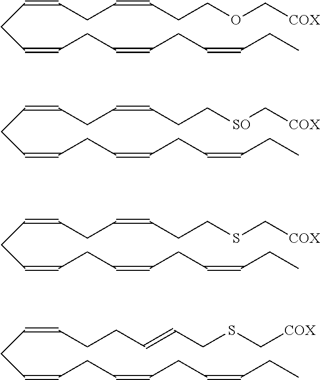
wherein X is as defined in claim 1, e.g. CF₃.

6. A compound for use as claimed in any preceding claim where the compound of formula (I) is Compound A1 or Compound CIX.

7. A method of preventing metastasis in cancer, especially breast cancer, comprising administering to a patient in need thereof, e.g. human, an effective amount of a compound of formula (I):
R-L-CO—X (I)
wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;
L is a linking group forming a bridge of 1 to 5 atoms between the R group and the carbonyl CO wherein L comprises at least one heteroatom in the backbone of the linking group; and
X is an electron withdrawing group; or a salt thereof.

8. Use of a compound of formula (I) or a salt thereof as described in claim 1 to 6 for use in the manufacture of a medicament for preventing metastasis in cancer, especially breast cancer.

9. A compound of formula (I)
R-L-CO—X (I)
wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;
L is a linking group forming a bridge of 1 to 5 atoms between the R group and the carbonyl CO wherein L comprises at least one heteroatom in the backbone of the linking group; and
X is an electron withdrawing group; or a salt thereof;
for use in a method of preventing metastasis in breast cancer comprising administering said compound to a patient with pre-metastatic breast cancer.
